(19) United States
(12) Patent Application Publication
(10) Pub. No.: US 2009/0142775 A1
(43) Pub. Date: Jun. 4, 2009

(54) Methods For Separating Organelles

(75) Inventor: May Tom-Moy, San Carlos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(73) Assignee: Agilent Technologies, Inc., Palo Alto, CA (US)

(21) Appl. No.: 11/948,101

(22) Filed: Nov. 30, 2007

(51) Int. Cl. G01N 33/53

(52) U.S. Cl. 435/7.2

(57) ABSTRACT

Methods and kits for separating organelles of a specific type from a mixture of organelles of different types are described. The methods comprise providing a mixture of organelles of different types, adding probes to the mixture to form an organelle-probe complex with a distinct diffusion coefficient, and separating the organelles of the specific type from the mixture of organelles based upon the diffusion coefficient of the complex. The probes used in these methods include an affinity portion which binds selectively to the organelle of the specific type and a tag portion which imparts the distinct diffusion coefficient to the complex.

### BACKGROUND

The study of organelle proteomics offers much promise in understanding cellular and biochemical systems. First, the targeting of proteins to particular subcellular sites is fundamental to understanding the functional organization of the cell at the molecular level. Second, by knowing the localization of a particular protein, one may be able to infer the function of that protein. Finally, the ability to monitor dynamic changes within the proteome may provide clues to protein translocation pathways which may precede changes in gene expression in disease states.

One of the challenges of proteome analysis is the detection of low copy number proteins. While the polymerase chain reaction provides a method for amplifying scarce DNA or RNA, there is no such comparable method for amplifying proteins. Accordingly, without fractionation of biological samples (e.g., specific organelles), highly abundant proteins will mask the identification of less abundant proteins. Moreover, many regulatory proteins of interest such as kinases, phosphatases, or GTPases are present in low copy numbers but have very specific subcellular localization. Accordingly, for these reasons and others, there is a need for improved methods to fractionate and analyze these organelles.

The prior art methods of subcellular fractionation typically consist of two major steps: (1) disruption of the cellular organization (homogenization) and (2) fractionation of the homogenate to separate the different populations of organelles.

Currently, most protocols to fractionate organelles rely on ultracentrifugation using either velocity sedimentation or equilibrium sedimentation. In velocity separations, particles move in the direction of the centrifugal force and separate according to size and density. Accordingly, it is time dependent as eventually all particles will pellet. In equilibrium separations, the particles move to a position in the gradient which reflects their respective buoyant densities. Centrifugation in step-gradients can enrich different compartments at interfaces between different sucrose concentrations. A problem with step-gradients is that small differences in density cannot be resolved. Alternatively, equilibrium separations with continuous gradients can be used. One particular drawback of continuous gradients can be the low yield of organelles. In either ultracentrifugation method, because organelles have similar densities, the organelles often overlap in the gradient, and thus the organelle fractions are often contaminated with organelles of a different type. This is an even bigger problem when one wishes to analyze the proteins of a particular type of organelle.

Other methods for fractionating organelles employ immunoisolation by identifying an appropriate antigen on the organelle of interest and preparing a high-affinity antibody that recognizes the antigen in its native state. The antibodies are then bound to solid supports such as columns made of cellulose (affinity chromatography) or magnetic beads. One problem with this method is that the cellulose fibers of the solid support can entrap and fragment the bound fractions resulting in low yields. Methods using columns and magnetic beads both suffer from cross-contamination of other organelles.

Free flow electrophoresis (FFE) is another method which separates organelles based on differences in the density of charge on the surfaces of membranes, independent of size and shape. In FFE, a mixture of differently charged organelles is injected into a buffer that flows between two charged plates, a cathode and an anode. However, it is difficult to obtain contaminant-free fractions of organelles due to the similarity in charge of many organelles.

Accordingly, there is a need for a method for separating pure fractions of specific types of organelles of interest. Such a method will be useful in better understanding the proteome of the cell.

### BRIEF DESCRIPTION OF THE DRAWINGS

FIG. 1 is a flow diagram that illustrates a method for separating organelles.

FIG. 2 is a schematic drawing that illustrates the formation of an organelle-probe complex comprising an organelle, an antibody, and a nanoparticle.

FIG. 3A is a schematic drawing that illustrates the operation of a field flow fractionator with an organelle-probe complex of the invention.

FIG. 3B is an enlarged cross-sectional view of FIG. 3A taken along line 3B-3B.

### DETAILED DESCRIPTION

Embodiments of the invention, as disclosed and described herein, provide a method for separating organelles of a specific type from a mixture of organelles of different types. FIG. 1 illustrates a method for separating organelles according to a typical embodiment of the invention. The method comprises providing a mixture of organelles of different types (box 1), adding probes to the mixture to form an organelle-probe complex with a distinct diffusion coefficient (box 2), and separating the organelles of the specific type from the mixture of organelles based upon the diffusion coefficient of the complex (box 3). In one embodiment of the invention, the method further includes lysing cells to release the mixture of organelles of different types.

The method may be used to separate any organelles of a specific type from a mixture of organelles of different types. In a typical embodiment, the organelles are obtained by lysing cells. For example, the organelles can be from prokaryotic or eukaryotic cells. The cells may be harvested from tissues or may be grown in culture. In a typical embodiment, the cells are yeast cells grown in culture. As used herein, the term “organelle” refers to a specialized subunit within a cell that has a specific function, and is separately enclosed within its own lipid membrane. Accordingly, the types of organelles may include nuclei, mitochondria, endoplasmic reticulum, Golgi apparati, vacuoles, lysosomes, peroxisomes, chloroplasts, acrosomes, autophagosomes, centrioles, ciliums, glycosomes, glyoxysomes, hydrogenosomes, melanosomes, mitosomes, myofibrils, nucleoli, parenthesomes, peroxisomes, ribosomes, or vesicles. The term “organelle” also encompasses other subcellular components including plasma membranes, flagellum, cilium, cell walls, cytoskeleton, and plasmodesmata. In a typical embodiment, the types of organelles include nuclei, mitochondria, endoplasmic reticulum, Golgi apparati, vacuoles, lysosomes, plasma membranes, or peroxisomes.

The probes, used in the methods of the present invention, include an affinity portion which binds selectively to a specific type of organelle. In a typical embodiment, the affinity portion of the probe is an antibody that has a high affinity to an appropriate antigen on the surface of the specific type of organelle in its native state. The antigen should be specific to a specific type of organelle or a subset of different types of organelles.

As used herein, the term “antibody” refers to a polypeptide or fragments thereof that specifically binds and recognizes an antigen. The term encompasses the immunoglobulin classes, IgG, IgM, IgA, IgD and IgE and the following types of antibodies: polyclonal, monoclonal, single-chain, humanized, chimeric antibodies, and fragments thereof. In a typical embodiment, the antibody is a monoclonal antibody. Hybridomas for the generation of monoclonal antibodies are prepared and screened using standard techniques. The methods used for monoclonal antibody development follow procedures known in the art.

Antibodies exist as intact immunoglobulins or as a number of well-characterized fragments produced by digestion with various peptidases. An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. While various antibody fragments are defined in terms of the digestion of an intact antibody, such fragments may be synthesized de novo either chemically or by using recombinant DNA methodology. Thus, the term antibody, as used herein, also includes antibody fragments either produced by the modification of whole antibodies, or those synthesized de novo using chemical or recombinant DNA methodologies.

In another embodiment, the affinity portion of the probe may be a protein ligand with a high affinity to a molecule on the surface of a specific type of organelle. As used herein, the term “ligand” refers to a molecule that binds to and forms a complex with a biomolecule. The biomolecule may be a receptor on the surface of a specific type of organelle or a subset of types of organelles.

The probes used in the methods of the invention include also a tag portion which imparts a distinct diffusion coefficient to the complex. In one embodiment, the tag portion is a microsphere. The microspheres are made of any suitable material, such as polystyrene or latex, that can be conjugated to the affinity portion of the probe. The microspheres are about 10 nm to about 200 μm, more typically about 20 nm to about 200 nm, and most typically about 50 nm to about 100 nm, in diameter. In another embodiment, the microspheres have activated surfaces optimized for either hydrophobic adsorption or covalent attachment of biological molecules. Examples of microspheres with activated surfaces include carboxylate-modified microspheres, aldehyde-modified micro spheres, carboxyl (hydrophobic) microspheres and sulfate (hydrophobic) microspheres. In one embodiment, the microspheres are aldehyde-modified. Typically, the microspheres are each conjugated to many antibodies in a one-step process. In another embodiment, the microspheres additionally include a dye such as a fluorescent dye incorporated into a polymer matrix.

In another embodiment, the tag portion is a nanoparticle. In one embodiment, the nanoparticle is made of gold. Other soft metals such as zinc may be substituted for gold. The nanoparticle is about 5 nm to about 200 nm, more typically about 20 nm to about 200 nm, and most typically about 50 nm to about 100 nm, in diameter.

The probe may also include a reporting agent. The term “reporting agent” refers to any peptide or small chemical molecule that can be used to identify the complex. In a particular embodiment, the reporting agent is a fluorophore, which is a functional group in a molecule which will absorb energy of a specific wavelength and re-emit energy at a different (but equally specific) wavelength. A common fluorophore is fluorescein isothiocyanate, a reactive derivative of fluorescein. Other common fluorophores are derivatives of rhodamine, coumarin and cyanine. In another embodiment, the reporting agent such as a dye is incorporated into the polymer matrix of the microspheres.

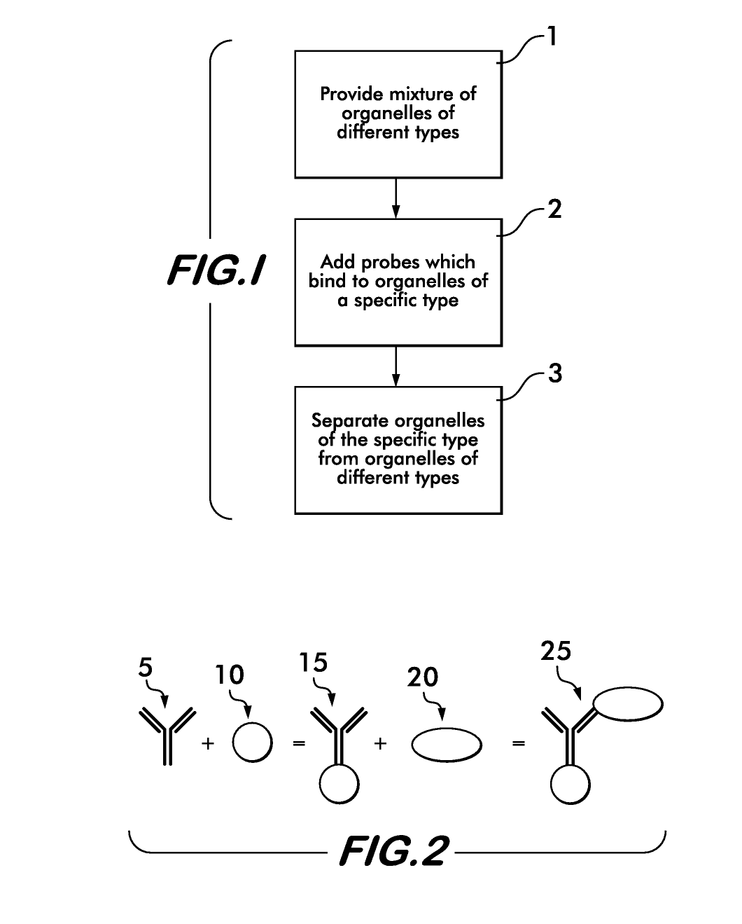

Referring to FIG. 2, in a typical embodiment, an antibody 5 is conjugated to a nanoparticle or microsphere 10 to form a probe 15 as described above or by standard methods known in the art. In a typical embodiment, the probes are purified to remove any unbound antibody or unbound tag by subjecting the probes to a vigorous wash step. The probes are then incubated with the mixture of organelles to allow binding of the probes to a specific type of organelle 20 to form an organelle-probe complex 25.

The mixture of organelles may comprise unpurified cell lysate. Alternatively, the cell lysate may first be “purified” by ultracentrifugation in a sucrose gradient. Fractions that contain an enriched population of the specific organelle of interest may then be used in the method. The process for forming the organelle-probe complex is schematically illustrated in FIG. 1.

The tag portion 10 imparts a distinct diffusion coefficient to the organelle-probe complex 25. As used herein, the term “diffusion coefficient” means the amount of a substance that diffuses through a unit area in unit time in response to a unit concentration gradient. The diffusion coefficient of the organelle-probe complex will vary depending on the size and mass of the tags.

A typical embodiment uses probes of two or more types. In such an embodiment, each type of probe binds selectively to a different type of organelle and imparts a respective distinct diffusion coefficient to the resulting organelle-probe complex. The distinct diffusion coefficient of the complex is the result of the type of probe having a tag that differs in at least one of size and mass from the tags of the other types of probes. A different reporting agent may be used with each type of probe. As discussed above, the method comprises separating the different types of organelles based upon the respective distinct diffusion coefficients of the organelle-probe complexes.

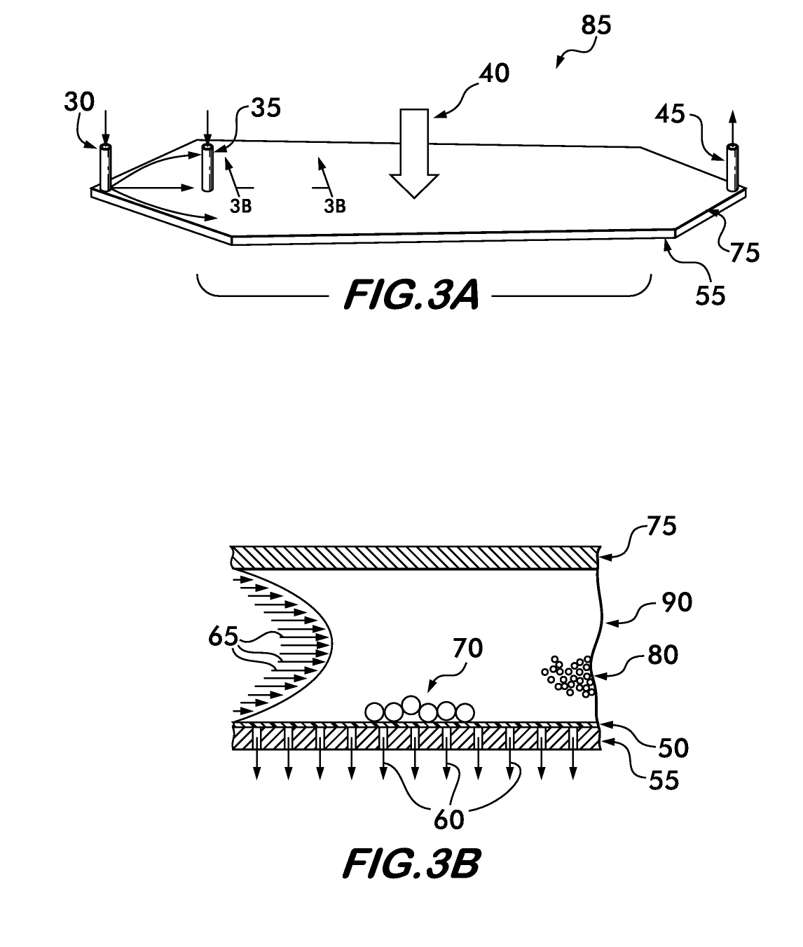

In a typical embodiment, the separating comprises injecting the mixture comprising the organelle-probe complexes into an asymmetric flow field flow fractionator (aFFF). Referring to FIGS. 3A and 3B, an aFFF 85 typically includes an impermeable channel plate 75 disposed opposite and parallel to a permeable channel plate 55 to define a channel 90. The permeable channel plate 55 is typically made of a perforated frit material. An ultrafiltration membrane 50 is juxtaposed with the permeable channel plate 55 between the permeable channel plate and the channel 90. The membrane 50 can have a molecular weight cut-off ranging from approximately 10 kDa to approximately 30 kDa. The aFFF 85 additionally typically includes an inlet port 30, an injection port 35, and an outlet port 45. Each of these ports extends through the upper channel plate 75 and is in fluid communication with the channel 90. A typical aFFF is approximately 30 cm long from the inlet port 30 to the outlet port 45, the channel 90 is approximately 4 cm wide and has a height ranging from approximately 100 μm to approximately 500 μm. Other channel dimensions may be used.

The separation process using aFFF 85 typically includes an injection process, a focusing process and an elution process. During the injection and focusing processes, an appropriate solvent is introduced into the channel through both the inlet port 30 and the outlet port 45. Examples of suitable solvents include 10 mM phosphate-buffered saline (PBS), pH 7.5, and Tris-HCl, pH 7.8. The rates of solvent flow through the inlet port 30 and the outlet port 45 are controlled such that the solvent flows meet at the injection port 35. In this state, the solvent additionally flows through the membrane 50 and permeable channel plate 55. This flow will be referred to as a cross-flow. The mixture of organelles and organelle-probe complexes is then injected into the channel 90 through the injection port 35. The contra-flow in the channel 90 of the solvent flow introduced through the inlet port 30 and that introduced through the outlet port 45 confines the mixture of organelles and organelle-probe complexes into narrow band that extends across the width of the channel. This confinement is known as “focusing.” Moreover, the cross flow of solvent through the permeable channel plate 55 increases the concentration of organelles and organelle-probe complexes towards the membrane 50.

After introduction of the mixture is complete, the elution process is performed. In this, the solvent flow pattern is switched to an elution mode in which the solvent enters the channel 90 only through the inlet port 30 and exits from the channel through the outlet port 45. The flow of solvent through the channel 90 from the inlet port 30 to the port outlet 45 is substantially laminar. The low height of the channel 90 creates a parabolic flow profile, schematically shown at 65, in which the flow is faster near the middle of the channel 90 than adjacent the channel plates (75, 55). As well as flowing along the channel 90, the solvent also flows towards the permeable channel plate 55 through which some of the solvent exits the channel. The solvent exiting the channel through the permeable channel plate 55 establishes a cross flow 60 that generates a force 40. The force 40 drives the mixture of organelles and organelle-probe complexes in the solvent towards the permeable channel plate 55. Organelles or organelle-probe complexes 80 that have a higher diffusion coefficient tend to reach an equilibrium position closer to the middle of the channel, where the solvent flow is faster. Organelles or organelle-probe complexes 70 that have a lower diffusion coefficient tend to reach an equilibrium position in the channel closer to the permeable channel plate 55, where the solvent flow is slower. As a result of this diffusion coefficient-dependent stratification into regions of different flow rate along the channel, the organelles elute from the outlet port 45 separately from the organelle-probe complexes because they have different diffusion coefficients. Moreover, when the mixture contains organelle-probe complexes of different types, the organelle-probe complexes of each different type elute from the outlet port 45 separately from one another and from the organelles in accordance with their respective diffusion coefficients. The organelle-probe complexes are collected at the outlet port in separate fractions.

In an embodiment, the method further comprises detecting the organelle-probe complexes that include the organelle of the specific type after separating such organelle-probe complexes from the mixture of organelles. A number of techniques can be used to detect the organelle-probe complex. If a fluorescent microsphere is used as the tag that constitutes part of the probe or if the probe additionally includes a fluorophore as an additional reporting agent, the complexes can be detected using an epifluorescence microscope, a confocal microscope, a fluorescence spectrophotometer, a fluorescence activated cell sorter, or by using other techniques or devices known in the art. If gold nanoparticles are used as the tag that constitutes part of the probe, the nanoparticle can be detected using a light scattering detector that can determine the size of any light scattering molecule or particle in the sample.

In a further embodiment of the invention, the method of separating organelles further comprises analyzing the protein composition of the organelle of the specific type. Fractions collected from the aFFF are processed for a number of different analyses including SDS-PAGE, 2D gel electrophoresis, microsequencing, western blots and mass spectrometry.

To analyze the collected fractions containing the organelle-probe complexes, the probe is dissociated from the respective bound organelle to provide a purified organelle. Dissociation of the probe from the organelle can be achieved through the use of one of several chaotropic reagents, such potassium or sodium thiocyanate, sodium chloride, trifluoroacetate, perchlorate, sodium iodide or guanidine-HCl.

The protein composition of the purified organelle can be separated by 2D gel electrophoresis. By fractionating subcellular organelles, an entire organelle-proteome can be displayed on a single 2D gel. As used herein, “2D gel electrophoresis” refers to a method for the separation of proteins in a sample by displacement in two orthogonal dimensions using two properties of the proteins (typically isoelectric point and mass). 2D gel electrophoresis is often used to isolate proteins for further characterization by microsequencing, mass spectroscopy or western blots.

Proteins from organelles may be eluted from the 2D gel and analyzed by microsequencing. “Microsequencing” refers to sequencing a portion of the amino acid sequence of a protein and using this information along with the estimated pI and molecular weight of the protein (from the gel) to identify the protein from a database. Protocols for performing microsequencing are well known in the art.

Western blots may be used to confirm if the fractions collected contain the specific organelle of interest. As used herein, a “western blot” refers to a method for detecting a protein of interest by first separating a mixture of proteins by gel electrophoresis, transferring the proteins to a membrane, and probing the proteins using antibodies specific to the protein of interest. Protocols for performing western blots are well known in the art.

Mass spectrometry may be used to identify specific proteins that are known to exist within that specific organelle of interest. Tandem mass spectrometry (MS/MS) is particularly useful for identifying proteins. Methods for fragmenting molecules for tandem MS that can be used include collision-induced dissociation (CID), electron capture dissociation (ECD), electron transfer dissociation (ETD), infrared multiphoton dissociation (IRMPD) and blackbody infrared radiative dissociation (BIRD).

In another embodiment, the invention comprises a kit for separating organelles of a specific type from a mixture of organelles of different types. The organelles subject to separation using the kit of this embodiment are as discussed above. The kit includes probes that have an affinity portion and a tag portion. The affinity portion binds selectively to organelles of the specific type to form an organelle-probe complex, and the tag portion imparts a distinct diffusion coefficient to the complex. In an alternative embodiment, the kit further includes probes of two or more types. Each type of probe binds selectively to a different type of organelle to form an organelle-probe complex and imparts a respective distinct diffusion coefficient to the resulting complex. In one embodiment, each type of probe is packaged separately. The probes of these embodiments are as discussed above. In a further embodiment, the kit further includes instructions for using the probes to separate organelles of a specific type from a mixture of organelles of different types using a field-flow fractionator.

### EXAMPLES

Part 1—Preparing Cell Lysate from Yeast Cells

Extracts are prepared from 8×10⁸ yeast cells, typically by overnight growth of a 100-ml culture to an OD₆₀₀ of 0.4. Cells are collected by centrifugation for 5 min at 500 g and are washed once in 10 mM solium azide, 10 mM potassium fluoride, and 50 mM Tris-HCl, pH 7.5, to poison energy-dependent processes. The cells are then are incubated in 0.8 ml of 100 mM ethylenediamionetetraacetic acid (EDTA), 0.5% 2-mercaptoethaneol, and 10 mM Tris-HCl, pH 7.5, for 20 min at 30° C. to reduce the disulfide bonds in the cell wall. The cells are then suspended in 0.8 ml S buffer (1.2 M sorbitol, 0.5 mM MgCl₂, 40 mM hydroxyethylpiperazine-N-2-ethanesulfonic acid (HEPES), pH 7.5) and are converted to spheroplasts by the addition of 50 U/OD₆₀₀ Zymolyase 100T (ICN Biomedicals, Inc., Costa Mesa, Calif.) for 45-60 min at 30° C. (I U Zymolyase will cause a decrease in OD₆₀₀ of 0.1 in 30 min at 30° C.). The extent of spheroplast formation is checked by recording the decrease in optical density when the spheroplast suspension is diluted into 1% Triton® X-100. Spheroplasting is complete when the optical density of the suspension in Triton X-100 has decreased to about 10% of that of spheroplasts in S buffer. Spheroplasts are washed once in S buffer and suspended in 1 ml lysis buffer (0.2 M sorbitol, 1 mM EDTA, and 50 mM Tris-HCl, pH 7.5). The spheroplasts are lysed by 20 strokes in a Dounce homogenizer with a tightly fitting pestle, with care to avoid introducing bubbles into the lysate. The extent of cell lysis can be checked by phase-contract microscopy. Unlysed cells and unmanageably large aggregates of organelles are removed by centrifugation at 500 for 5 min at 4° C. The clear lysate (CL) is collected.

### Part 2—Optional Purification of Organelles

A linear sucrose gradient (30-60%) is made using a gradient maker. The CL from Example 1 is layered on the bottom of the gradient and centrifuged in a model L-80XP ultracentrifuge (Beckman Coulter, Inc., Fullerton, Calif.) using a SW32.1 rotor for 19 hours at 100,000 g. Subsequently, 1 ml fractions are collected and stored at 4° C. until use.

### Part 3—Selecting Antibodies for Binding to Organelles

Antibodies are selected that bind to the type or types of organelles for which further investigation is desired. Hybridomas for the generation of monoclonal antibodies are prepared and screened using standard techniques. The methods used for monoclonal antibody development follow procedures known in the art such as those detailed in Kohler and Milstein, Nature 256: 494 (1975) and reviewed in J. G. R. Hurrel, ed., Monoclonal Hybridoma Antibodies: Techniques and Applications, CRC Press, Inc., Boca Raton, Fla. (1982). Another method of monoclonal antibody development which is based on the Kohler and Milstein method is that of L. T. Mimms et al., Virology 176: 604-619 (1990), which is incorporated herein by reference. Alternatively, these antibodies are commercially available from vendors such as Invitrogen Corporation, Carlsbad, Calif. or other antibody suppliers.

### Part 4—Conjugating Antibodies to Microspheres to Form Probes

The following general procedure outlines the conjugation of antibodies to aldehyde-modified polystyrene or latex microspheres to form the probes used in embodiments of the invention. Other types of microspheres designed to attach to biological molecules may also be used. Examples include carboxylate-modified microspheres or sulfate microspheres.

A 50 mM reaction buffer at pH 7.0-7.5 is prepared and used to prepare a 10 mg/ml solution of the antibodies in the reaction buffer. Several different antibody concentrations and reaction buffers at different pH should be tried in a series to optimize binding of the antibodies to the microspheres. A 1% w/v suspension of aldehyde-modified microspheres in reaction buffer is prepared. Aldehyde-modified microspheres may be purchased from Duke Scientific Corporation and range in size from 0.10 μm to 0.80 μm and are supplied as aqueous suspensions at 4% solids, The microspheres may be purchased from other suppliers in other sizes and also are available with high intensity colored or fluorescent dyes. One volume of the antibody solution is added rapidly to ten volumes of the microsphere suspension with very efficient mixing, such as with a vortex mixer. If the reaction is carried out in volumes too large to use a vortex mixer, the microsphere suspension should be vigorously stirred using a magnetic stirrer in a flask or beaker and the antibody solution added rapidly to the center of the vortex. The mixture is then incubated at room temperature for 2 hours. Unbound antibodies are then removed from the mixture. If microsphere conjugates are greater than 0.2 μm in diameter, the microsphere conjugates can be washed by centrifugation. The pellet comprising the probes is resuspended between washes by vortexing followed by gentle ultrasonication using a bath sonicator. The probes may also be purified by filtration methods. The probes may be stored in an appropriate buffer until used

### Part 5—Conjugating Antibodies to Nanoparticles to Form Probes

First, the minimum amount of antibodies required to stabilize the gold nanoparticles is determined 0.25 ml of the nanoparticle gold suspension is added to separate tubes containing 25 μl of different concentrations of the antibodies to be adsorbed and mixed well. The amount of antibodies required to stabilize 1 ml of most gold sols is in the microgram range. The antibody concentrations should be from about 10 μg/100 μl to about 150 μg/100 μl. After about 1 min, 0.25 ml of 10% NaCl is added to the gold/antibody suspension and mixed well.

The stability of the gold nanoparticle solution is monitored by its color or by the absorbance of the mixture at 580 nm. As long as the colloid continues to turn blue, and thus form gold aggregates, with addition of electrolyte, the amount of antibodies added is not sufficient to stabilize the suspension. This condition translates into a decrease in the absorbance at 580 nm. When the concentration of antibodies added is enough to stabilize the colloidal suspension, the solution no longer changes color, or the absorbance at 580 nm no longer decreases.

A stabilizing amount of antibodies plus an additional 10% is mixed with the appropriate volume of gold nanoparticle suspension. The colloidal suspension should be adjusted, if needed, with 0.1 M K₂CO₃ or NaOH to pH 8-9. After 1 minute, a quantity of 10% bovine serum albumin (BSA) is added to bring the concentration to 0.25% in the antibody-gold suspension. The BSA helps to further stabilize the sol against aggregation and also blocks nonspecific binding sites. Alternatively, polyethylene glycol (PEG) may be added. The sol is stirred for an additional 5 minutes. To remove excess antibodies, the preparation is centrifuged at a minimum of 50,000 g for 30 minutes to several hours at 4° C., depending on the size of the particles and the amount of solution. The supernatant is discarded and the probes are re-suspended in 0.01 M sodium phosphate, pH 7.4, containing 0.25% BSA (or 1% PEG, as desired).

Part 6—Labeling Antibodies with Fluorophore

The amount of reactive dye is determined by using the manufacturers directions. A 1 M sodium bicarbonate solution is prepared by adding 1 ml deionized water to the appropriate amount sodium bicarbonate. 20-100 μl of a 1 mg/ml solution of antibodies (20-100 μg) is transferred to a reaction tube. 2-10 μl of 1 M sodium bicarbonate is added and mixed by pipetting up and down several times. The appropriate volume of reactive dye solution is added to the reaction tube containing the pH-adjusted antibody solution and mixed thoroughly by pipetting up and down several times. The reaction mixture is incubated for 15 minutes at room temperature.

The unreacted dye is separated from the labeled antibodies. To prepare for separating the labeled protein from unreacted dye, the upper chamber of a spin filter is filled up to the lip with suspended gel resin (which is typically supplied by the manufacturer of the reactive dye); approximately 800 μl of resin will be needed. The spin filter is centrifuged at 16,000 g in a microcentrifuge for a total of 15 seconds (including run-up time) to create a resin bed.

If desired, the buffer in which the resin is supplied may be exchanged by decanting or aspirating the buffer in which the resin is supplied while in its storage bottle, replacing with a desired buffer, mixing to resuspend the resin, and allowing to settle. This washing process is repeated several times. Alternatively, the desired buffer may be washed through the resin bed by brief low-speed centrifugation.

After the spin filter is prepared, no more than 50 μl of the conjugate reaction mixture is pipetted onto the center of the resin bed surface. If the volume of conjugate reaction mixture is 51-100 μl, it is divided into two aliquots and purified on separate spin filters. The spin filter(s) are placed in the microcentrifuge with the high side of the resin bed on the outside and centrifuged at 16,000 g for a total of 1 minute. The purified dye-labeled antibodies in approximately 60-100 μl of buffer are contained in the collection tube(s). The unreacted dye is retained in the filter.

The conjugate of labeled antibodies is stored at 2-6° C. and protected from light. It may be necessary to add a stabilizer like BSA (1-10 mg/ml) or glycerol to the conjugate to improve stability. In the presence of 2 mM sodium azide or other biocides, a typical antibody conjugate should be stable at 2-6° C. for several months. Alternatively, the conjugate may be separated into small aliquots and frozen at ≧−20° C. for longer storage.

### Part 7—Binding Probes to Organelles

The probes from Part 4 or Part 5 are incubated with either the clear lysate (CL) from Part 1 or fraction(s) of the sucrose gradient from Part 2 to form probe-organelle complexes of at least one type. The sucrose gradient fraction from Part 2 contains an enriched population of the specific organelle of interest with other different types of organelles that have similar buoyant densities. Probes ranging in concentration from 0.05 to 4 μg/ml are incubated with 1-2 μg/ml of the CL or sucrose gradient fractions in 1.2 M sorbitol, 0.5 mM MgCl₂, and 40 mM HEPES buffer, pH 7.5. BSA at a concentration ranging from 0.05% to 1% is also added in the incubation to prevent non-specific adsorption of the probes to non-organelle particulates. The incubation is for 1-24 hours at 4° C.

Following incubation, the organelle-probe complexes may be purified to remove unbound probes from solution. Known purification protocols such as size exclusion or affinity chromotography are used to remove the unbound probes. For example, an Econo-Pac® Protein A column (Bio-Rad Laboratories, Inc, Hercules, Calif.) may be used to purify the complexes. A column is equilibrated with 10 ml binding buffer. The buffer is allowed to drain to the top frit. After equilibration, the pH of the column effluent should be equal to the pH of the binding buffer (pH 9.0). No more than 2 ml of the prepared sample comprising the organelle-probe complexes is applied to the column. The column is washed with 20 ml of binding buffer. The organelle-probe complexes are eluted with 10 ml of elution buffer. The column is eluted with an additional 20 ml of buffer to ensure total removal of the organelle-probe complexes. One to two ml fractions are collected from the column and absorbance is monitored using a diode array spectrophotometer or fluorimeter to identify the purified organelle-probe complex fractions.

Part 8—Fractionating the Organelle-Probe Complexes Using an Asymmetric Field-Flow Fractionator (aFFF)

An Eclipse aFFF sold by Wyatt Technology Corp. (Santa Barbara, Calif.) is used to fractionate the mixture comprising the organelle-probe complexes. The aFFF is first validated according to the manufacturer's instructions. Validation typically includes checking the focusing with a dye solution and running a standard sample. First, the aFFF is primed and flushed to ensure that the aFFF is free of bubbles. Next, separate flows of solvent are introduced into the channel 90 through the inlet port 30 and the outlet port 45. The solvent flow rates through the ports are controlled such that the solvent flows meet under the injection port 35. A standard sample (e.g., BSA, latex standard particles, etc. is then injected into the injection port 35 and the solvent flow rates through the inlet port 30 and the outlet port 45 are controlled to focus the standard sample into a thin band with a greater concentration of the standard sample towards the membrane 50. The focusing parameters are checked and adjusted as necessary according to the manufacturer's instructions. Typically, the aFFF is maintained in this state for another minute before the solvent flow pattern is switched to the above-described elution flow mode in which the solvent enters the channel 90 only through the inlet port 30, the main flow of solvent exits the channel through the outlet port 45 and the cross flow of solvent exits the channel through the permeable channel plate.

For fractionating organelle-probe complexes, in the injection process, the injection flow through the injection port 35 is at 0.20 ml/min. In the focusing process, the solvent flow rate through both the inlet port 30 and the outlet port is at 1.0 ml/min. In the elution process, the flow rate of solvent into the inlet port 30 is varied between 1.0 ml/min and 6 ml/min, the flow rate of solvent, organelles and organelle-probe complexes from the outlet port 45 is maintained at 1.0 ml/min, and the cross flow rate is varied between 0.5 ml/min and 5 ml/min. Fractions are collected from the outlet port of the aFFF using a thermostated fraction collector that collects fractions from time 0 min to time 45 min, which marks the end of the separation run. The volume of the fraction collected is dependent on the flow rate. Typical volumes range from 0.5 ml to 4.5 ml per fraction.

If a fluorescent microsphere is used as the tag portion of the probes or a fluorophore is used as an additional label, the emission of the fluorophore or fluorescent microsphere can be monitored during the separation and those peaks that would represent the specific organelle with which the antibody portion of the probe binds can be collected. If gold nanoparticles are used as the tag portion of the probes, the nanoparticle can be detected using a light scattering detector that can determine the size of any light scattering molecule or particle in the sample.

The collected fractions collected are then processed for a number of different analyses including protein analysis, SDS-PAGE, 2D gel electrophoresis, microsequencing, western blots and mass spectrometry using well known protocols in the art.

Part 9—Dissociating the Probe from Organelle-Probe Complex

The organelle-probe complexes at a concentration of approximately 100 μg/μl are incubated with 6 M guanidine-HCl pH 4.0 for 5, 10 or 20 minutes at room temperature. The released probes are separated from the organelles by size exclusion chromatography using PD-10 Micro Bio-Spin™ columns from Bio-Rad Laboratories, Inc. (Hercules, Calif.) or Econo-Pac® Protein A columns (described in Part 7). The released probes will bind to the Protein A and the organelles will be present in the eluant. The eluant containing the organelle fraction may be analyzed using Western blots or Mass Spectrometry.

### Part 10—Preparing Organelle Samples for SDS-PAGE and Western Blots

SDS-PAGE—Organelle samples at 1 μg/μl are incubated with an equal volume of Laemmli buffer (62.5 mM Tris-HCl, 2% SDS, 25% glycerol, and 0.01% bromophenol blue, pH 6.8) and incubated for 5 min at 95° C. The digested sample is then loaded onto a 10.5%-14% Tris-HCl polyacrylamide gel. The current applied for the electrophoresis is 35 mA/gel for 55 min.

Western Blot—The Bio-Rad® Criterion™ system with 40% Tris-Glycine/40%-Tris-SDS/20% MeOH is used to transfer proteins to Bio-Rad inmun-Blot® Nitrocellulose or PVDF membranes at 175 mA/membrane for 3 hours. Membranes are blocked for one hour with 5% non-fat dry milk and incubated overnight with specific monoclonal antibodies (MAbs) in 1% BSA and PBS/0.1% Tween-20. MAbs specific to markers of the plasma membrane (Pma1p), cytoplasm (PgK1p) endoplasmic reticulum (Dpm1p), and Golgi and late endosome (Pep12p) are used. Such MAbs may be purchased from Invitrogen Corp. (Carlsbad, Calif. or Abcam plc whose US office is located in Cambridge, Mass. Goat anti-mouse IgG-HRP is used as the secondary antibody. The Opti-4CN substrate kit and the Strepavidin amplification kit from Bio-Rad is used to detect peroxidase activity.

### Part 11—Preparing Organelle Samples for MS/MS

Approximately 10-20 μg of protein in 100 mM ammonium bicarbonate is dried using a speed vac. The dried protein is resuspended in 100 μl of 50% 100 mM ammonium bicarbonate buffer, pH 8.0/2,2,2-trifluoroethanol (TFE) (product 326747 sold by Sigma-Aldrich, Inc.). 5 μl of dithiothreiothol (DTT) is added and the suspension is incubated at room temperature for 60 min. 600 μl HPLC grade water is then added followed by adding 200 μl of 100 mM ammonium bicarbonate, pH 7.8. 20 μl of 200 mM iodoacetamide is added, and the suspension is mixed and incubated in the dark for 60 min. 10 μl of Trypsin (1 μg) is added, and the suspension is further incubated at 37° C. for 18-14 hours. 5 μl of formic acid is added. The digested protein in the suspension is dried by speed vac and then resuspended in 50 μl of 0.1% formic acid.

The peptides are analyzed using an HPLC-Chip LC-MSD XC Ultra Trap MS sold by Agilent Technologies, Inc. (Santa Clara, Calif.). The separation is performed on a protein ID chip (G4240-62001) with a 40-nl enrichment column, 43 mm×75 μm analytical column, packed with ZORBAX® 300SB C18 reverse-phase material. Samples are eluted using a linear gradient of 2-60% acetonitrile, 0.1% formic acid at a flow rate of 250 nl/min. The mass spectrum data is analyzed using the Agilent® G2721AA Spectrum Mill® MS Proteomics Workbench software and searched against the NCBlnr database.

The invention is not to be limited in scope by the specific embodiments described herein. Indeed, various modifications of the invention in addition to those described herein will become apparent from the foregoing description. Such modifications are intended to fall within the scope of the appended claims.

It is further to be understood that all values are approximate, and are provided for description.

All patents, applications, publications, test methods, literature, and other materials cited herein are hereby incorporated by reference.

1. A method of separating organelles of a specific type from a mixture of organelles of different types, the method comprising:
providing said mixture of organelles of different types;
adding to said mixture probes comprising an affinity portion and a tag portion, wherein said affinity portion binds selectively to said organelles of said specific type to form an organelle-probe complex, and said tag portion imparts a distinct diffusion coefficient to said complex; and
separating said organelles of said specific type from said mixture of organelles based upon said diffusion coefficient of said complex.

2. The method of claim 1, wherein said providing comprises providing a cell comprising said mixture of said organelles of said different types, and lysing said cell to release said mixture of said organelles of said different types.

3. The method of claim 2, wherein said cell is a eukaryotic cell.

4. The method of claim 2, wherein said cell is a yeast cell.

5. The method of claim 1, further comprising detecting said complex.

6. The method of claim 1, further comprising analyzing the protein composition of said organelles of said specific type.

7. The method of claim 1, wherein said adding comprises adding probes of two or more types to said mixture, wherein each type of probe binds selectively to a different type of organelle to form a respective complex and imparts a respective distinct diffusion coefficient to said complex.

8. The method of claim 1, wherein said types of organelles are selected from the group consisting of nucleus, mitochondrion, endoplasmic reticulum, Golgi apparatus, vacuole, lysosome, plasma membranes, and peroxisomes.

9. The method of claim 1, wherein said affinity portion of said probe is an antibody that binds selectively to said organelles of said specific type.

10. The method of claim 1, wherein said affinity portion of said probe is a ligand that binds selectively to said organelles of said specific type.

11. The method of claim 1, wherein said tag portion of said probe is a microsphere.

12. The method of claim 11, wherein said microsphere is a polymer microsphere.

13. The method of claim 11, wherein said microspheres includes a dye.

14. The method of claim 13, wherein said adding comprises adding probes of two or more types to said mixture, and wherein said each type of probe includes a different dye.

15. The method of claim 11, wherein said microsphere is about 10 nm to about 200 μm in diameter.

16. The method of claim 1, wherein said tag portion of said probe is a nanoparticle.

17. The method of claim 16, wherein said nanoparticle is a gold nanoparticle.

18. The method of claim 16, wherein said nanoparticle is about 5 nm to about 200 nm in diameter.

19. The method of claim 1, wherein said probes additionally comprise a reporting agent.

20. The method of claim 19, wherein said reporting agent is a fluorophore.

21. The method of claim 1, wherein said separating comprises injecting said mixture into an asymmetrical field flow fractionator.

22. A kit for separating organelles of a specific type from a mixture of organelles of different types, said kit comprising probes, said probes comprising an affinity portion and a tag portion, said affinity portion binding selectively to organelles of said specific type to form an organelle-probe complex, and said tag portion imparting a distinct diffusion coefficient to said complex.

23. The kit of claim 22, further comprising probes of two or more types, wherein each type of probe binds selectively to a different type of organelle to form a respective complex and imparts a respective distinct diffusion coefficient to said complex.
